(19) United States
(12) Patent Application Publication
(10) Pub. No.: US 2021/0063387 A1
(43) Pub. Date: Mar. 4, 2021

(54) MEANS AND METHOD FOR DETECTING ANALYTES BY MEANS OF MACROSCOPIC GRANULATE PARTICLES

(71) Applicant: AJ Innuscreen GmbH, Berlin (DE)

(72) Inventors: Timo Hillebrand, Hoppegarten (DE); Elmara Graser, Berlin (DE)

(73) Assignee: AJ Innuscreen GmbH, Berlin (DE)

(21) Appl. No.: 16/641,951

(22) PCT Filed: Aug. 26, 2017
(86) PCT No.: PCT/EP2017/071479

§ 371 Date: Oct. 28, 2020

(51) Int. Cl. G01N 33/543

(52) U.S. Cl. CPC G01N 33/54326

(57) ABSTRACT

The invention relates to magnetically separable polymer-based macro granules for carrying out immunoassays for detecting highly diverse analytes for medical, biological and biotechnological sectors. The size of the macroscopic granulate particles is between 0.5 mm and 10 mm in cross-section, preferably between 1 mm and 5 mm. Preferably, the macro granules can be magnetically separated. According to a preferred embodiment, the polymer-based macro granules are located in a pipette tip.

The present invention relates to the use of a magnetically separable polymer based macro granule for carrying out immunoassays for detecting highly diverse analytes for medical, biological and biotechnological sectors.

### STATE OF THE ART

Apart from the molecular-biological diagnostics, analysis and detection of proteins, antibodies etc. have become an indispensable component in modern medical laboratory diagnostics, forensic diagnostics, veterinary laboratory diagnostics, and in food diagnostics and in environmental diagnostics.

Immunoassays and their derivatives are always being used, if the analyte does not exist in the form of a nucleic acid which could be reproduced for detection by molecular-biological methods. This relates to the detection of different molecules such as, for example, hormones, toxins, alcohols and proteins in general. Moreover, immunoassays are being used as so-called quick tests, when a rapid detection, for example, of an entire cell or a virus particle is sought. These detection technologies are among others ELISAs (for the detection of proteins or nucleic acids, R. Yalow et al. J. Clin. Invest. 39, 1157-75 (1960), blotting techniques (for the detection of proteins and nucleic acids, Southern, E. M. J. Mol. Biol. 98 503-517 (1975); Alwine J C et al. Proc. Natl. Acad. Sci. U S. A 74, 5350-5354 (1977): Renart, J. et al. Proc. Natl. Acad. Sci. U S. A. (7), 3116-3120 (1979), biochip techniques (for the detection of proteins and nucleic acids, for example, WO 8808875), lateral flow test strips (for the detection of proteins and nucleic acids, for example, U.S. Pat. No. 4,956,302) or the detection by means of bio barcodes (proteins and nucleic acids US 20030054358 A1). As detection molecules, not only antibodies but also aptamers, enzymes (for example alcohol test) or individual, chemically active molecules (such as, for example, nitrite test) are being used. But many of the detection methods for proteins are coupled to expensive, device-dependent systems (ELISA reader, electrophoreses, blotter, washer, etc.), and require at the same time a time-consuming methodology (ELISA, western blotting, chip based detection). On the contrary, lateral flow assays constitute a simple alternative, which can be rapidly carried out, but with respect to their sensitivity, they can often be used only in a limited way.

The sensitivities, which can be achieved, constitute a general problem of the detection technologies presented. On the one hand, this is due to the signal amplification, which is only possible in a limited way, on the other hand, it is due to the small amount of sample which can be used for the corresponding test format (in the case of a conventional ELISA 100 μl max.). Added to this are still problems with possible matrix effects in the case of difficult sample materials, such as, for example, blood plasma, foodstuff etc. These problems are partly solved by the use of different low cross buffers [1]. By means of these buffers, the original sample is diluted, and added to an antibody. This increases specificity of the antibody-antigen binding but results in a loss of sensitivity due to the dilution of the starting material.

Previous technologies for solving said problem regarding the limited sample volume provide for the use of beads to which the analyte is bound [2]. Here, the size of the beads used is in the micrometer and nanometer sector. At first, this appears to be very advantageous, because beads have a very large surface at a small total volume. The beads coupled with antibodies are being added to the sample, and subsequently supplied to the further detection method together with the antigen bound on it, either by a centrifugation or by magnetic separation. Moreover, beads are also being used for the separation of various sample components, such as, for example, exosomes, cells and molecules. The separation of the beads from a solution, however, is always difficult, because in particular beads in the micrometer and/or submicrometer scale require a very long time in order to be separated by means of a magnetic field, even more so, the more viscous the sample to be examined is. Therefore, a loss of beads during magnetic separation, or a very long separation time must always be expected. When larger sample volumes are being used, these problems increase accordingly. This is the reason why the total reaction volume in these methods is limited to 1 to 2 ml. Another problem occurs by the magnetic properties of such beads, which is the reason for an aggregation of the beads which might lead to a loss of the assay functionality during storage.

An advantage could be offered by so-called macro particle based ELISA. The use of functionalized microparticles for ELISA of a size of approx. 0.6 cm has already been published [4]. However, the microparticles known until now do not have any magnetic and/or paramagnetic properties. Therefore, this does not permit a magnetic separation of the beads either for carrying out the detection reaction. Another problem is the lack of possibility of automation of macro particle based detection methods. A very interesting development regarding an ELISA automation is the use of a modified pipette tip [3]. Here, the pipette tips themselves have been coated with an antibody. The subsequent ELISA has been carried out on the surface of the tips as a solid phase. The self-contained pipette tips provide a protection against cross-contamination, but the usable volume of the sample material, however, is very limited here (30 μl of sample are being used by the authors). Moreover, this methodology requires a very complicated apparatus, and a very complex production of the functionalized tips, and can therefore not be used for diagnostic routine applications in a cost-effective/cost-covering manner.

### Task of the Invention

The task of the present invention is defined by the disadvantages mentioned of known detection systems.

### Solution of the Task

The task has been solved in accordance with the features of the patent claims.

The means according to the invention permits a rapid signal generation, where appropriate, from a large sample volume, without complicated equipment technology, a very high sensitivity, and, moreover, is suitable for an automated process of any type whatsoever. It represents an alternative, novel means for carrying out previous ELISA applications.

The means according to the invention for the detection of an analyte uses specific macroscopic granulate particles (‘MPG’). These particles can have different shapes and sizes, preferably sizes between 1 mm and 5 mm in cross-section. The particles can consist of the known materials which have the capacity to carry on their surface the functioning detection molecules (antibodies, aptamers, chemical groups etc.). Moreover, the granulate used can be separated by means of a magnet.

Such a material is known as a granulate under the brand name TECACOMP®.

The means according to the invention also has the coatable surface required for ELISA. After coating has been carried out, the granulate according to the invention can be used for performance of the detection reaction. This is based on the procedures known for the ELISA applications:
1. A surface coated granulate is brought into contact with a sample which contains an analyte to be detected. This can occur by the fact that the granulate is located freely in the sample or by leading the sample past the granulate. The analyte is specifically binding to the granulate in that case.
2. After a short incubation, the granulate is separated from the sample by means of magnetic separation, and subsequently briefly washed.
3. Then, the granulate is brought into contact with detection molecules (e.g. HRP marked antibodies).
4. Thereafter, the granulate is washed again by using magnetic separation in order to efficiently separate unbound detection molecules.
5. The final detection occurs, e.g., after the addition of a substrate solution and by colorimetric measurement of the substrate turnover reaction.

The analyte bound to the means according to the invention is acting as a bridge between the means according to the invention and the other detection components. Detection components may be marked molecules here, which on the one hand specifically bind to the analyte, and on the other hand permit the possibility of a detection (e.g. horseradish peroxidase marked antibodies, aptamer marked with a fluorescent substance, another detection molecule coupled to particles which contain the detection molecules themselves etc.).

In accordance with the objective of the present invention, the means according to the invention is suitable in a most ideal way to permit a detection reaction. The granulate has a sufficiently large binding surface for the analyte. Due to its macro properties and its magnetic properties, it can be used in a very large sample volume (e.g. 1 ml to 100 ml). For this purpose, one preferably uses a process in which the sample is lead past the means according to the invention. As a result, the analytes contained in the sample are enriched on the surface of the granulate in an accumulating way. This approach permits on the one hand to increase the sensitivity of the process, and on the other hand to dilute the sample with the analyte with a more favorable buffer for the binding, and thus minimize the matrix effects of the sample. Another important advantage of the means according to the invention as compared with a bead based ELISA application consists in the fact that due the size of the granulates, the separation of the granulates from the sample occurs very rapidly. In contrast, the beads of a nanometer and micrometer size require a very long time in order to be able to be separated. Losses of beads do no longer occur at all which is a considerable problem for small beads.

A special embodiment for automation of the detection method consists of a pipette tip which is filled with the granulate according to the invention. Pipetting and pipetting off of the sample permits the desired liquid fluctuation on the surface of the means according to the invention. By means of pipetting steps, the granulate can also be brought into contact with the detection components. On the other hand, if required, the granulate can be brought into contact with a large sample volume already prior to transfer to the tip, and can be transferred to the tip after magnetic separation which is not possible when a pipette tip is used as a solid phase in an ELISA [3].

When the granulate is transferred to a pipette tip, its magnetic properties step back in significance, but its macroscopic properties can be used: MGP remain in the pipette tip during the pipetting steps. Transfer of the granulate into a pipette tip also solves the problem of cross-contamination of adjacent samples. It is also possible to use all automation systems, which are based on magnetic separation, for the ELISA to be performed which is not possible with non-magnetic macro particles. Furthermore, separation of the granulates by means of a magnetic field can also occur in a pipette tip.

The means according to the invention is particularly suitable, for example, for use in an online monitoring in flow systems. Here, the binding of the analyte to the means according to the invention occurs within a bypass line, and the analyte can subsequently be detected within a short period of time outside the line.

The invention shall be described more in detail below by means of examples. The embodiments shall not limit the invention, though.

### EMBODIMENT 1

### Detection of the C Reactive Protein (CRP)

The detection limit of CRP in commercial assays is approx. at 1 mg/L. A polypropylen magnetic granulate (diameter approx. 4 mm) has been incubated for 5 hours with anti CRP antibody (Senova GmbH). Due to the hydrophobic interactions, the antibody attached to the granulate surface. Subsequently, the functionalized granulate was blocked. One functionalized granule each was used per detection reaction. The second anti CRP antibody was conjugated with HRP (Horseradish peroxidase) for a later colorimetric detection.

A dilution series of the CRP antigen has been produced:

1: 30 mg/l, 2: 3 mg/l, 3: 1.5 mg/l, 4: 0.75 mg/l, 5: 0.37 mg/l, 0.06 mg/l

The immunoassay has been carried out as follows:
1. A magnetic granule was brought into contact with 50 μl CRP antigen from the dilution series and 150 μl PBS.
2. Incubation 30 min under rotational movement.
3. 3× washing with conventional ELISA-Washing-Buffer (AJ Robescreen GmbH), accompanied by magnetic separation of the magnetic granule.
4. Addition of the HRP marked anti CRP detection antibody to the granule.
5. Incubation 30 min under rotational movement.
6. 3× washing with conventional ELISA-Washing-Buffer (AJ Robescreen GmbH), accompanied by magnetic separation of the magnetic granule.
7. The magnetic granules were transferred into a ELISA reader compatible microtiter plate.
8. A colorimetric HRP mediated TMB staining served for measuring of the samples in an ELISA reader at 450 nm and a reference wavelength of 630 nm (Thermo Fisher). Measuring results (mean value from 3× reaction) see table 1.

TABLE 1
Measuring values at 450 nm

| | | |
|---|---|---|
| No | Conzentration | Measuring value OD^{450 nm} |

| | | | |
|---|---|---|---|
| 1 | 30 | mg/l | 2.859 |
| 2 | 3 | mg/l | 1.488 |
| 3 | 1.5 | mg/l | 0.725 |
| 4 | 0.75 | mg/l | 0.374 |
| 5 | 0.37 | mg/l | 0.185 |
| 6 | 0.06 | mg/l | 0.097 |

| | | |
|---|---|---|
| 7 | No Ag | 0.060 |
| 8 | ELISA BG | 0.055 |

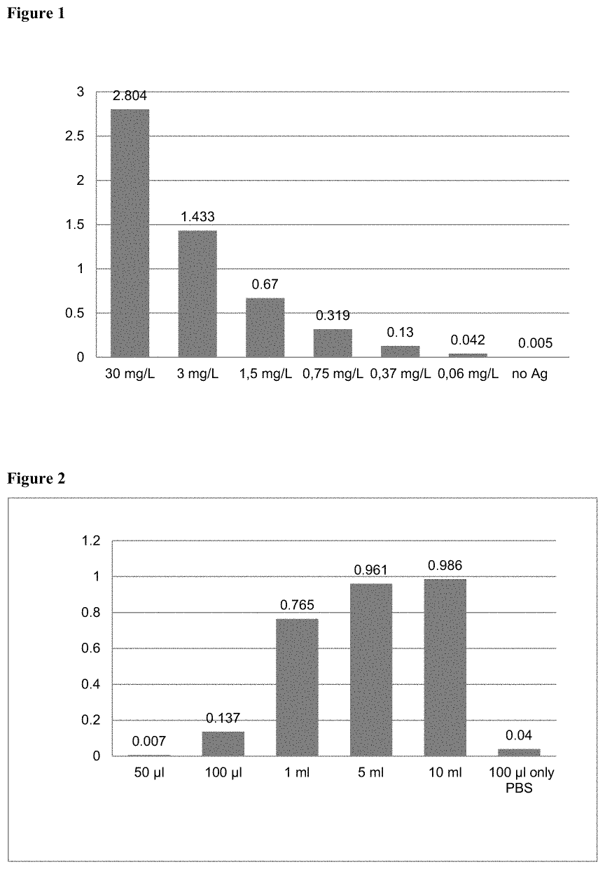

FIG. 1 shows the presentation of the measuring values less background control.

The test demonstrates a low background detection of the CRP antigen up to detection limits which are below the detection limit of conventional CRP ELISA.

### EMBODIMENT 2

### Impact of Sample Volume on Signal Strength at CRP Detection

Preparation of the magnetic granule for the test occurred as described in the embodiment 1.

The CRP antigen has been produced in a concentration of 0.03 mg/l.

The magnetic granules have been incubated with the following volumes of the sample:

1. 50 μl, 2. 100 μl, 3. 1 ml, 4. 5 ml, 5. 10 ml

After an incubation for one hour, the granules have been prepared for a detection with an ELISA reader as in example 1. The results of the measurement see table 2 (mean value from 3×determination)

TABLE 2
Measuring values at 450 nm

| | | |
|---|---|---|
| No | Volumes | Measuring value OD450 nm |

| | | | |
|---|---|---|---|
| 1 | 50 | μl | 0.062 |
| 2 | 100 | μl | 0.192 |
| 3 | 1 | ml | 0.82 |
| 4 | 5 | ml | 1.016 |
| 5 | 10 | ml | 1.041 |

| | | |
|---|---|---|
| 6 | 100 μl only PBS | 0.095 |
| 7 | ELISA BG | 0.055 |

FIG. 2 shows the presentation of the measuring values less blank.

The test shows that the increase of the sample volume leads to a higher sensitivity, which constitutes an advantage compared with a conventional ELISA, in which the sample volume is limited by the size of the microtiter plate.

### BIBLIOGRAPHY

[1] http://www.candor-bioscience.de/methoden/elisa.html
[2] A new method of measuring C-reactive protein, with a low limit of detection, suitable for risk assessment of coronary heart disease.
Eda S, Kaufmann J, Molwitz M, Vorberg E.
Scand J Clin Lab Invest Suppl. 1999; 230:32-5.
[3] An automated ELISA system using a pipette tip as a solid phase and a pH-sensitive field effect transistor as a detector Hitoshi Tsuruta, Hideaki Yamada, Yukiko Motoyashiki, Keiko Oka, Chieko Okada, Michihiro Nakamura Journal of Immunological Methods 183 (1995) 221-229
[4] Vadim V Shmanai, Tamara A Nikolayeva, Ludmila G Vinokurova, and Anatoli A Litoshka Oriented antibody immobilization to polystyrene macrocarriers for immunoassay modified with hydrazide derivatives of poly(meth)acrylic acid BMC Biotechnol. 2001; 1: 4

1: A means for detecting an analyte, comprising:
magnetic macroscopic granulate particles, comprising
a surface coating to which the analyte to be detected specifically binds.

2: The means according to claim 1, wherein the size of the macroscopic granulate particles is between 0.5 mm and 10 mm in cross-section.

3: The means according to claim 1, wherein the macroscopic granulate particles comprise a conglomeration between a polymer and a magnetic and/or paramagnetic material, and can therefore be magnetically separated.

4: The means according any one of claim 1, wherein antibodies, aptamers or chemically functional groups serve as a surface coating.

5: A test kit for detecting an analyte, comprising,
the means according to claim 1 a washing buffer, a detection molecule and a means for visualization of the detection process.

6: A device for detecting an analyte, comprising:
at least a pipette tip in which macroscopic granulate particles according to claim 1 are located.

7: The test kit for detecting an analyte, comprising:
the device according to claim 6 a washing buffer, a detection molecule and a means for visualization of the detection process.

8: The test kit according to claim 5, wherein said test kit is an ELISA.

9: An automatic device for detecting an analyte, comprising:
several pipette tips according to in which magnetic macroscopic granulate particles are located, the macroscopic granulate particles comprising a surface coating to which the analyte to be detected specifically binds,
containers with the sample of the analyte, washing buffers, detection molecules and means for visualization of the detection process,
wherein the pipette tips are immersed according to the walk-away principle one after the other into the containers of the analyte, the washing buffers and the detection molecules, and finally the substrate-turnover-reaction is visualized on a suitable means.

10: A method for detecting an analyte, the method comprising:
a) contacting macroscopic magnetic granulate particles, comprising a surface coating to which the analyte to be detected specifically binds,
with a sample, which contains an analyte to be detected,
wherein the analyte is bound to the granulate particles which are either located freely in the sample or by leading the sample past the granulate,
b) after a short incubation, separating the granulate from the sample by
magnetic separation, and subsequently briefly washing,
c) subsequently, contacting the granulate with detection molecules,
d) thereafter, washing the granulate again by use of magnetic separation in order to separate unbound detection molecules,
e) finally detecting the analyte after addition of a substrate solution and by measuring of the substrate-turnover-reaction on a suitable means and/or a direct measurement of the marked detection molecules.

11: A method for online monitoring in a flow system, the method comprising:
contacting an analyte with a means for detecting an analyte according to claim 1.
